(19) United States
(12) Patent Application Publication
(10) Pub. No.: US 2025/0040978 A1
(43) Pub. Date: Feb. 6, 2025

(54) MULTIPOLAR-TYPE OUTPUT APPARATUS AND CONTROL METHOD THEREFOR

(71) Applicant: WONTECH CO., LTD, Daejeon (KR)

(72) Inventors: Jong Won KIM, Seongnam (KR); Jung Hyun KIM, Seongnam (KR); Young Seok SEO, Sejong (KR)

(21) Appl. No.: 18/920,885

(22) Filed: Oct. 19, 2024

(30) Foreign Application Priority Data
Jan. 22, 2004 (KR) 10-2024-0133842

(51) Int. Cl. A61B 18/00

(52) U.S. Cl. CPC A61B 18/00; A61B 2018/00452

(57) ABSTRACT

Proposed are a multipolar-type output apparatus and a control method for the multipolar-type output apparatus. The multipolar-type output apparatus includes a body, a handpiece, and a tip. The tip comprises an electrode unit configured to transmit the high frequency energy received from the handpiece to skin, wherein the electrode unit comprises a first electrode and a second electrode, the first electrode and a second electrode having a size different from each other

### CROSS REFERENCE TO RELATED APPLICATION

The present application claims priority to Korean Patent Application No. 10-2024-0133842, filed Oct. 2, 2024, the entire contents of which are incorporated herein for all purposes by this reference.

### BACKGROUND

### Technical Field

The present disclosure relates to a multipolar-type output apparatus and a control method therefor. More particularly, the present disclosure relates to a multipolar-type output apparatus and a control method for the multipolar-type output apparatus capable of outputting high frequency energy both in a bi-polar irradiation method and in a mono-polar irradiation method with a single tip.

### Description of the Related Art

Recently, a technology in which energy is provided to skin by using various energy sources to treat the skin by transforming a state of tissue of the skin or by improving tissue characteristics is widely applied. Skin treatment apparatuses using various energy sources such as a laser beam, a flash lamp, a supersonic wave, and so on have been developed, and recently, researches about a skin treatment apparatus using high frequency (RF) energy has been actively conducted.

When high frequency energy is provided to a skin surface, molecules constituting skin tissue vibrate and rub each other every time a current direction of high frequency changes, so that deep heat is generated due to rotation, torsion, and collision of the molecules. Such deep heat increases a temperature of the skin tissue and reorganizes a collagen layer, so that wrinkles can be decreased and skin elasticity can be reinforced. Furthermore, blood circulation in the skin tissue is increased and accelerated, so that overall state of the skin is improved, including skin anti-aging.

In order to achieve the effect as described above, there is a technology of irradiating the skin with high frequency energy to deeply penetrate into the skin. When the skin is irradiated with high frequency energy, a mono-polar irradiation method or a bi-polar irradiation method may be used. The mono-polar irradiation method is a method in which the same current is output and transmitted, and the bi-polar irradiation method is a method in which two different currents are output and transmitted. In the mono-polar irradiation method, either positive (+) or negative (−) current is selected, the selected current is output through a single electrode, the selected current is transmitted below the skin, and the mono-polar irradiation method has a larger transmission depth than a transmission depth of the bi-polar irradiation method. In addition, in the mono-polar irradiation method, a return electrode is required to be provided so as to allow current to flow. On the other hand, in the bi-polar irradiation method, since a positive (+) electrode and a negative (−) electrode are provided, current flows from the positive electrode to the negative electrode, so that the bi-polar irradiation method has the transmission depth lower than the transmission depth of the mono-polar irradiation method and the bi-polar irradiation method does not require a return electrode. The mono-polar irradiation method is efficient for a deep skin layer for realizing wrinkle removal and so on, and the bi-polar irradiation method is efficient for a shallow skin layer for collagen regeneration and so on.

However, since a tip for performing the mono-polar irradiation method and a tip for performing the bi-polar irradiation method are separately provided, two tips are required to be provided for performing treatment with each irradiation method, and there is a problem that the cost of skin care treatment is increased.

In order to solve this problem, there is a technology capable of performing both the mono-polar irradiation method and the bi-polar irradiation method with a single tip. However, since the mono-polar irradiation method is performed with an electrode for performing the bi-polar irradiation method, there is a problem that an RF energy does not reach a required transmission depth or the RF energy is small and the effect is reduced during irradiating with the RF energy.

### SUMMARY

Accordingly, the present disclosure has been made keeping in mind the above problems occurring in the related art. However, the above problems are provided only for the better understanding of the present disclosure and do not limit the scope of the inventions of this application.
and an objective of the present disclosure is to provide a multipolar-type output apparatus and a control method for the multipolar-type output apparatus in which electrode placements on a tip surface in contact with skin are configured such that a size of a first electrode unit and a size of a second electrode unit are different from each other and an output electrode of the first electrode is capable of being switched, thereby being capable of performing both a mono-polar irradiation method and a bi-polar irradiation method and being capable of performing a single irradiation operation and a continuous irradiation operation.

In order to achieve the objective described above, according to the present disclosure, there is provided a multipolar-type output apparatus including: a body configured to generate a high frequency energy; a handpiece detachably attached to the body and configured to transmit the high frequency energy received from the body to the tip; and a tip detachably attached to the handpiece and comprising an electrode unit configured to transmit the high frequency energy received from the handpiece to skin, wherein the electrode unit comprises a first electrode and a second electrode, the first electrode and a second electrode having a size different from each other.

The tip may comprise a contact surface at a portion of the tip that is brought into contact with the skin, and the first electrode may be in a protruding shape protruding from the contact surface of the tip, while the second electrode is in a flat shape.

The first electrode may be in a form of a needle to invade into the skin.

The first electrode may be in a form of a mild protrusion not to invade into the skin.

The first electrode may comprise a plurality of first electrodes, each of which being in the protruding shape.

The second electrode, which is in the flat shape, may be provided in an area surrounding the plurality of first electrodes.

The plurality of first electrodes and the second electrode may be distanced away from each other on the contact surface of the tip such that the plurality of first electrodes and the second electrode are not electrically connected on the contact surface of the tip.

The first electrode may comprise a first group of multiple first electrodes and a second group of multiple first electrodes, and the second electrode may comprise at least two second electrodes.

One of the at least two second electrodes may be provided in an area surrounding the first group of multiple first electrodes, and the other of the at least two second electrodes may be provided in an area surrounding the second group of multiple first electrodes.

The body may comprise an energy generation unit configured to generate high frequency energy transmitted to the electrode unit and a body controller configured to control a polarity of the high frequency energy.

The body controller may be operable to set the polarity of the high frequency energy in at least two different irradiation modes, in which the high frequency energy having different polarities depending on the at least two irradiation modes, according to a user input.

The at least two different irradiation modes may comprise: a first irradiation mode, in which the first group of multiple first electrodes have a positive (+) polarity while the second group of multiple first electrodes have a negative (−) polarity, and a second irradiation mode, in which the first group of multiple first electrodes, the second group of multiple first electrodes, the at least two second electrodes have a same polarity, either positive (+) or negative (−).

In the first irradiation mode, the at least two second electrodes may be controlled not to irradiate with the high frequency energy.

The body may comprise a cooling unit, and the handpiece may include a gas transferring unit configured to receive a cooling gas generated from the cooling unit of the body and to transfer the cooling gas to the tip, and the tip may comprise a chamber where the cooling gas supplied from the gas transferring unit stays when the cooling gas is output.

The body may comprises a body controller and a storage storing a first identification value, and the tip may include a second storage unit storing a second identification value of the tip, wherein the body controller may determine matching of the first identification value and the second identification.

The handpiece may comprise a handpiece controller configured to set operation of the handpiece in at least two different operation selections, the at least two different operation selections comprise a first operation selection in which the electrode unit performs one time irradiation operation and a second operation selection in which the electrode unit performs continuous.

According to the present disclosure, there is provided a control method for a multipolar-type output apparatus comprising a body, a handpiece, and a tip with an electrode unit, wherein the electrode unit comprises a plurality of first electrodes and at least two second electrodes, the at least two second electrodes having a different size from the plurality of first electrodes, the control method including: determining, by a controller, whether a first irradiation mode or a second irradiation mode is detected, a first irradiation mode, in which a first group of multiple first electrodes from among the plurality of first electrodes have a positive (+) polarity while a second group of multiple first electrodes from among the plurality of first electrodes have a negative (−) polarity, and a second irradiation mode, in which the first group of multiple first electrodes, the second group of multiple first electrodes, the at least two second electrodes have a same polarity, either positive (+) or negative (−); generating high frequency energy from an energy generation unit of the body and outputting the high frequency energy to the electrode unit of the tip through the handpiece so as to irradiate skin with the high frequency energy according to detected first irradiation mode or second irradiation mode.

The first irradiation mode may be a high frequency energy transmission method in a bi-polar irradiation method, and the second irradiation mode may be a high frequency energy transmission method in a mono-polar irradiation method.

The control method may further include changing, by the controller, between a first operation selection and a second operation selection, a first operation selection in which the electrode unit performs one time irradiation operation and a second operation selection in which the electrode unit performs continuous irradiation.

The control method may further include generating a small amount of high frequency energy from the energy generation unit first and irradiating the skin with the small amount of high frequency energy so as to determine whether a return electrode is properly provided and outputting the high frequency energy to the electrode unit of the tip through the handpiece so as to irradiate the skin with the high frequency energy when it is determined that the return electrode is properly provided.

The return electrode may be electrically connected to the body, and a data value of the return electrode may be recognized by the controller.

Whether the return electrode is properly provided may be determined on the basis of a resistance value input to the controller.

The control method may further include displaying a warning window on a display unit that the return electrode is not properly provided when the resistance value exceeds 400Ω.

According to these features, since the size and the placement of the first electrode and the second electrode are different, an area of electrodes irradiating the skin is increased, so that there is an effect that the energy transmission is increased and the energy transmission depth is increased and there is an effect that a cumbersome need to use two tips is reduced.

In addition, due to the switching of the positive polarity and the negative polarity of the first electrode, the area of the first electrode may be utilized when the mono-polar irradiation method is performed.

In addition, by changing the function of the second control unit according to the irradiation mode, there is an effect that the single irradiation operation and the continuous irradiation operation are capable of being performed.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a configuration of a multipolar-type output apparatus according to an embodiment of the present disclosure;

FIGS. 2A to 2C are schematic views illustrating electrode placements of a tip of the multipolar-type output apparatus according to embodiments of the present disclosure;

FIG. 3 is a flowchart illustrating a control method for the multipolar-type output apparatus according to an embodiment of the present disclosure;

FIG. 4 is a perspective view illustrating a configuration of the multipolar-type output apparatus according to an embodiment of the present disclosure.

### DETAILED DESCRIPTION

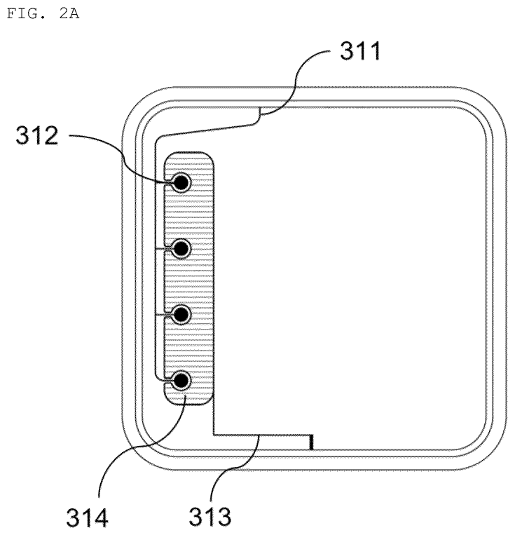

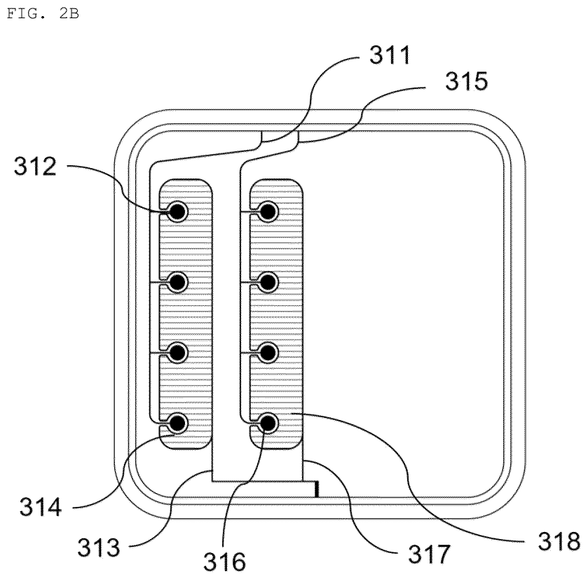

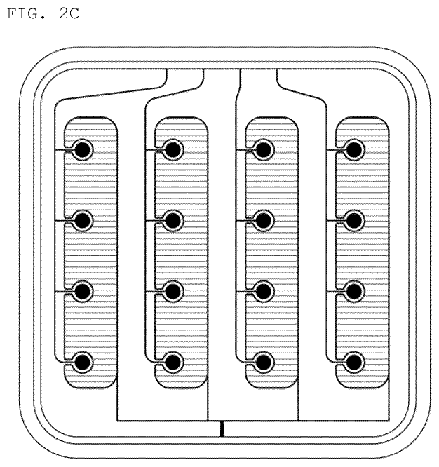

Hereinafter, an embodiment of the present disclosure will be described in detail with reference to the accompanying drawings such that a person having ordinary knowledge in the technical field to which the present disclosure belongs may easily implement the embodiment. However, it is understood that the present disclosure is able to be implemented in various different forms and is not limited to the embodiments described herein. In addition, in the following description of the present disclosure, detailed descriptions of known functions and components incorporated therein will be omitted if they are deemed to obscure the gist of the subject matter of the present disclosure. Also, elements in drawings, especially in FIGS. 2A, 2B, and 2C, the element which are apparently similar according to the drawings, are depicted to indicate multiple presence of those similar elements in corresponding positions.

Throughout the specification, when a part is referred to as being “connected” (connect, contact, combine) to another part, it includes being “directly connected” to another part and “indirectly connected” to another part with still another part disposed therebetween. In addition, unless explicitly described to the contrary, the word “comprise” and variations such as “comprises” or “comprising” will be understood to imply the inclusion of stated elements but not the exclusion of any other elements.

The terminology used herein is for the purpose of describing particular embodiments only and is not intended to limit the present disclosure. As used herein, the singular forms are intended to include the plural forms as well, unless the context clearly indicates otherwise. In addition, it will be further understood that the terms “comprise”, “include”, “have”, and so on when used in the present application, specify the presence or absence of stated features, integers, steps, operations, elements, components, and/or combinations of them but do not preclude the possibility of the presence or addition of one or more other features, integers, steps, operations, elements, components, and/or combinations thereof.

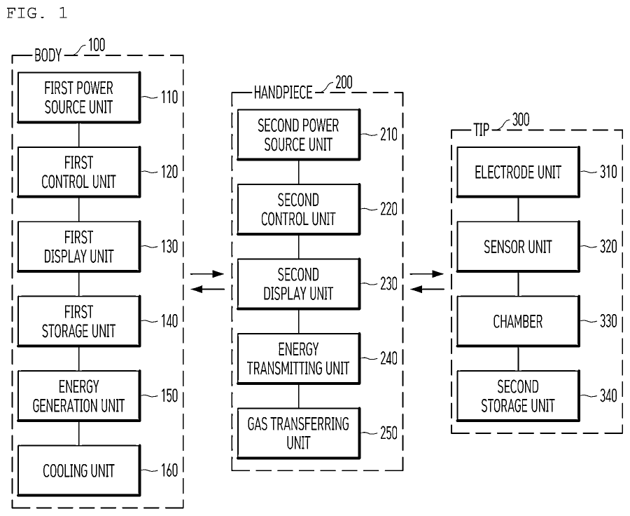

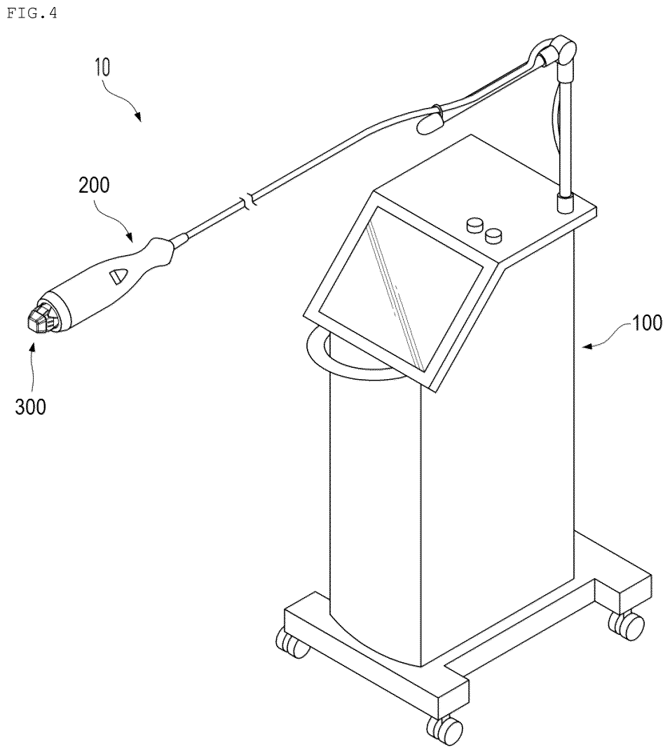

Then, a multipolar-type output apparatus and a control method for the multipolar-type output apparatus according to an embodiment of the present disclosure will be described with reference to FIG. 1, FIGS. 2A to 2C and FIG. 4.

FIG. 1 is view illustrating a configuration of the multipolar-type output apparatus according to an embodiment of the present disclosure, and FIGS. 2A to 2C are schematic views illustrating electrode placements of a tip of the multipolar-type output apparatus according to an embodiment of the present disclosure; and FIG. 4 is a perspective view illustrating a configuration of the multipolar-type output apparatus according to an embodiment of the present disclosure.

Referring to FIG. 4, the multipolar-type output apparatus may include a tip 300, a handpiece 200, and a main body 300. The tip 300 may be referred to as simply a tip 300, and the body 100 may be referred to as simply a body 100.

The body 100 generates high-frequency energy (e.g., high-frequency current) of a predetermined frequency required for skin treatment. The frequency of the high-frequency energy may be determined differently depending on a patient's treatment purposes or treatment sites.

The body 100 may include an operation unit for controlling the power or frequency of the device 10. The body 100 may further include a user interface for receiving a user input and displaying various information. The displayed information may include status of the device 10, patient information and operating the device 10. The user interface may be a touch screen.

The handpiece 200 is a component having the tip 300 attached to an end part thereof, and is configured to provide high-frequency energy for skin treatment to the tip 300. Specifically, the handpiece 200 receives the high-frequency energy from the main body 100, transmits the high-frequency energy to the tip 300, and controls high-frequency output and operation of the tip 300. The handpiece 200 may be in a generally elongated shape in a longitudinal direction. The handpiece 200 may be connected to the body 100 at its proximal end and has the tip 300 at its distal end.

As one exemplary embodiment, an outer housing of the handpiece 200 may be provided with operation buttons, and a user may control the multipolar-type output apparatus, such as turning the pressing unit on/off, manipulating a penetration depth of the needle array, or turning the high-frequency energy output of the tip 300 on/off through the operation button. At the distal end of the handpiece 200, the outer housing of the handpiece 200 may include a distal end plate.

The tip 300 may include the needle array. The needle array, by moving up-and-down in the longitudinal direction, may be exposed to the outside or stored inside the housing. For example, during treatment, the needle array is moved out and exposed to the outside of the Tip 300 and penetrates into the patient's skin, and when the treatment is completed, the needle array is retracted into the tip 300.

The tip 300 has a structure detachably attachable to the handpiece 200, receives high-frequency energy from the handpiece 200, and emits the high-frequency energy to the inside of the skin through the needle array.

Referring to FIG. 1 and FIGS. 2A to 2C, the multipolar-type output apparatus according to an embodiment may include: a body 100 configured to perform an overall operation and configured to generate high frequency energy; a handpiece 200 which is capable of being electrically connected to the body 100 and detachably attached to the body 100, the handpiece 200 being configured to transmit high frequency energy; and a tip 300 which is capable of being electrically connected to the handpiece 200 and detachably attached to a distal end of the handpiece 200, the tip 300 being configured to irradiate with high frequency energy.

The body 100, the handpiece 200, and the tip 300 in FIG. 1 correspond to the body 100, the handpiece 200, and the tip 300 in FIG. 4, respectively.

The body 100 may include: a first power source unit 110 configured to supply power; a first control unit 120 configured to receive power from the first power source unit 110 and to control overall operations of the body 100, the handpiece 200, and the tip 300; a first display unit 130 configured to receive power from the first power source unit 110 and to display an operation state to the multipolar-type output apparatus and a user; a first storage unit 140 configured to receive power from the first power source unit 110 and to be operated by being controlled by the first control unit 120, the first storage unit 140 storing data matching with unique data (e.g., an identifier) of the tip 300; an energy generation unit 150 configured to receive power from the first power source unit 110 and to be operated by being controlled by the first control unit 120, the energy generation unit 150 being configured to generate high frequency energy; and a cooling unit 160 configured to receive power from the first power source unit 110 and to be operated by being controlled by the first control unit 120.

Each of the first power source unit 110, the first control unit 120, the first display unit 130, the first storage unit 140, the energy generation unit 150, and the cooling unit 160 may be in hardware with an integrated circuit or in a combination of hardware and software.

The handpiece 200 may include: a second power source unit 210 configured to receive power from the first power source unit 110 of the body 100 and to transmit an overall power for the handpiece 200; a second control unit 220 configured to receive power from the second power source unit 210 and to be linked with the first control unit 120, the second control unit 220 being configured to control operations of the body 100, the handpiece 200, and the tip 300; a second display unit 230 configured to receive power from the second power source unit 210 and to display operation states of the first control unit 210 and the second control unit 220 to the user; an energy transmitting unit 240 configured to receive power from the second power source unit 210 and to receive high frequency energy generated from the energy generation unit 150 of the body 100 and to transmit the high frequency energy to the tip 300; and a gas transferring unit 250 configured to receive a cooling gas generated from the cooling unit 160 of the body 100 and to transfer the cooling gas to the tip 300.

The first control unit 120 of the body 100 and the second control unit 220 of the handpiece 200 may perform all or at least some of the other's functions. Each of the first control unit 120 of the body 100 and the second control unit 220 of the handpiece 200. The processor in the first control unit 120 and/or the processor may include a processor with an integrated circuit. Also, each of the body 100 and the handpiece 200 may include a memory to store programs. The processors in the first control unit 120 and/or the second control unit 220 may perform the functions of the first control unit 120 and the second control unit 220 to control the entire operations of the multipolar-type output apparatus 10 according to the instruction in the program.

The tip 300 may include: an electrode unit 310 coupled to and electrically connected to the handpiece 200, the electrode unit 310 being configured to receive high frequency energy generated from the energy generation unit 150 of the body 100 through the energy transmitting unit 240, and the electrode unit 310 being configured to transmit high frequency energy to the skin of the patient; a sensor unit 320 configured to receive power from the second power source unit 210 and to measure biometric data of the patient; a chamber 330 where the cooling gas supplied from the gas transferring unit 250 stays when the cooling gas is output; and a second storage unit 340 storing the unique data (e.g., an identifier) of the tip 300 and storing data matching with the first storage unit 140 of the body 100.

According to an embodiment, the unique data stored in the first storage unit 140 of the body 100 may match with the unique data in the second storage unit 340 of the tip 300. The matched unique data may include a unique identification number for determining whether the tip 300 coupled to the handpiece 200 is a genuine product manufactured by a same manufacturer of the body 100.

An operation function of the second control unit 220 of the handpiece 200 may be controlled by the control of the first control unit 120 and vice versa. The first control unit 120 and/or the second control unit 220 may control the multipolar-type output apparatus 10 to operate in any one of a single irradiation operation t operation selection) and a continuous irradiation operation (a second operation selection). The first control unit 120 and/or the second control unit 220 may identify a user input to determine an operation selection, either the single irradiation operation (a first operation selection) or a continuous irradiation operation (a second operation selection). That is, whether to perform the single irradiation operation or the continuous irradiation operation of the high frequency energy generated in the energy generation unit 150 according to operation detection of the second control unit 220 may be changed by the first control unit 120 and/or the second control unit 220.

The energy generation unit 150 of the body 100 may generate either a positive (+) polarity or a negative (−) polarity, may generate both polarities simultaneously. The type of polarity generated is determined by the control of the first control unit 120.

The electrode unit 310 of the tip 300 is formed on a portion that is brought into contact with the skin of the patient. According to an embodiment, the tip 300 may include a contact surface at the portion that is brought into contact with the skin of the patient. The contact surface of the tip 300 may be generally perpendicular to the longitudinal direction of the handpiece 200 when the tip 300 is coupled with the handpiece 200. The electrode unit 310 may be provided on the contact surface of the tip 300.

As illustrated in FIG. 2A, the electrode unit 310 includes: a first transmitting unit 311 configured to transmit high frequency energy to a first electrode 312 by being controlled by the first control unit 120; the first electrode 312 connected to the first transmitting unit 311 and configured to irradiate with high frequency energy transmitted through the first transmitting unit 311; a second transmitting unit 313 configured to transmit high frequency energy to a second electrode 314 by being controlled by the first control unit 120; and the second electrode 314 connected to the second transmitting unit 313 and to irradiate with high frequency energy transmitted through the second transmitting unit 313. The first transmitting unit 311 and the second transmitting unit 313 may be conductive wires through which current flows.

The plurality of first electrodes 312 protrudes from the contact surface of the tip 300 in a longitudinal direction. The plurality of first electrodes 312 may be in form of a needle to invade into the skin of the patient. Alternatively, the plurality of first electrodes 312 may be in form of a mild protrusion not to invade into the skin. The number of first electrodes 312 that protrude may be one to six, but is not limited thereto.

The second electrode 314 may be in a generally flat form on the contact surface of the tip 300. According to an embodiment, multiple second electrodes 314 may be provided on the contact surface of the tip 300. The second electrode 314 may be provided in an area surrounding at least one of the first electrodes 312, while the first electrodes 312 and the second electrode 314 are distanced away from each other on the contact surface of the tip 300 so that they are not electrically connected on the contact surface of the tip 300. Therefore, the first electrode 312 and the second electrode may be in a different size, in a different shape and/or in a different arrangement.

Referring to FIG. 2B, a group of first electrodes 312, each of which is in a protruding shape, may be generally surrounded by the second electrode 314, which is in a flat shape, while a group of third electrodes 316, each of which is in a protruding shape, may be generally surrounded by a fourth electrode 318, which is in a flat shape.

In addition, as illustrated in FIG. 2C, multiple sets of the first transmitting units 311, the first electrodes 312, the second transmitting units 313, and the second electrodes 314 may be provided on the contact surface of the tip 300.

The body 100 may further include a switching unit (not illustrated). The switching unit may control the energy generated in the energy generation unit 150 to have either positive (+) or negative (−) polarity according to an operation of the first control unit 120 and/or the second control unit 220. The body 100 and/or the handpiece 200 may receive a user input to select a bi-polar irradiation mode (a first irradiation mode) or a mono-polar irradiation mode (a second irradiation mode).

Referring to FIG. 2B, when irradiation is performed in a bi-polar irradiation method (the first irradiation mode), the first transmitting unit 311 may transmit a high frequency energy of the positive (+) polarity, and the third transmitting unit 315 has the negative (−) polarity. In addition, at this time, according to an embodiment, the second transmitting unit 313 and a fourth transmitting unit 317 may be controlled by the switching unit of the body 100 so that the second transmitting unit 313 and a fourth transmitting unit 317 do not irradiate with high frequency energy.

Therefore, in the first irradiation mode, the first electrode 312 irradiates with high frequency energy of the positive (+) polarity from the first transmitting unit 311, while the third electrode 316 has the negative (−) polarity from the third transmitting unit 315, so that the energy of the positive (+) polarity output from the first electrode 312 flows to the third electrode 316 having the negative (−) polarity. At this time, the energy is transmitted while passing through a shallow portion of the skin layer.

On the other hand, referring to FIG. 2B, when irradiation is performed in a mono-polar irradiation method (the second irradiation mode), the first control unit 310 and/or the second control unit 320 may control the switching unit so that at least one of the first transmitting unit 311, the second transmitting unit 313, the third transmitting unit 315, and the fourth transmitting unit 317 unify and transmit high frequency energy with a same polarity, either positive (+) or negative (−) polarity, while none of them transmit high frequency energy with an opposite polarity.

Also, the tip 300 may have a vibration unit (not illustrated) provided on one side of the tip 300. The vibration unit may be configured to be operated by a control of the first control unit 120 and/or a control of the second control unit 220. Furthermore, since vibration is directly transmitted to the user's skin through the vibration unit, there is an effect of alleviating pain.

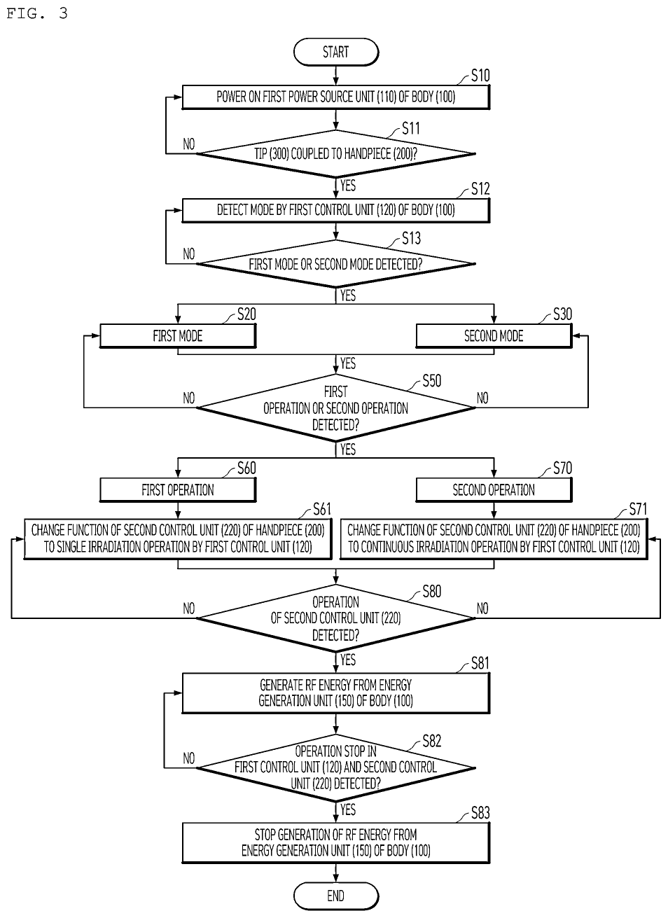

Next, a control method for the multipolar-type output apparatus according to an embodiment of the present disclosure will be described with reference to FIG. 3.

FIG. 3 is a flowchart illustrating the control method for the multipolar-type output apparatus according to an embodiment of the present disclosure. The steps in the flow chart of FIG. 3 may be performed under a control of the Referring to FIG. 3, in step S10, power is applied to the first power source unit 110 of the body 100 first (S10).

When power is applied to the first power source unit 110, whether the tip 300 is coupled to the handpiece 200 is determined (S11).

When it is determined that the tip 300 is coupled to the handpiece 200 in step S11, an irradiation mode is detected by the first control unit 120 of the body 100 (S12).

Then, in step S13, the first control unit 120 determines which of a first mode (i.e., the bi-polar mode or a first irradiation mode) or a second mode (i.e., the mono-polar mode or a second irradiation mode) is detected (S13). Here, the first mode is a high frequency energy transmission method in the bi-polar irradiation method, and the second mode is a high frequency energy transmission method in the mono-polar irradiation method.

In the process of determining whether the first mode or the second mode is detected (S13), if it is determined that the first mode is detected (S20), the first control unit 120 determines which of a first operation (i.e. a first operation selection) or a second operation (i.e., a second operation selection) is detected (S50).

If the first operation is detected in step S60, the first control unit 120 sets a function of the second control unit 220 of the handpiece 200 to a single irradiation operation (S61).

After the function of the second control unit 220 is set to the single irradiation operation (S61), whether an operation for start of the second control unit 220 is detected is determined (S80). In other words, in step S80, it is determined whether there is an input to start frequency energy generation.

When it is determined that the operation for start of the second control unit 220 is detected in step S80, high frequency energy is generated from the energy generation unit 150 of the body 100, and the multipolar-type output apparatus 10 irradiates the skin with the high frequency energy by outputting the high frequency energy to the electrode unit 310 of the tip 300 through the handpiece 200.

Then, in step S82, the first control unit 120 and/or the second control unit 220 determine whether an operation for stop is detected (S82).

When the operation for stop is detected in the first control unit 120 and/or the second control unit 220 in step S82, the generation of high frequency energy is stopped at the energy generation unit 150 of the body 100 (S83).

In the process of determining whether the first operation or the second operation is detected (S50), if the second operation (S70) is detected, the first control unit 120 sets the function of the second control unit 220 of the handpiece 200 to a continuous irradiation operation (S71).

After the function of the second control unit 220 is set to the continuous irradiation operation (S71), whether the operation for start of the second control unit 220 is detected is determined (S80).

When it is determined that the operation for start of the second control unit 220 is detected in step S80, high frequency energy is generated from the energy generation unit 150 of the body 100, and the multipolar-type output apparatus 10 irradiates the skin with the high frequency energy by outputting the high frequency energy to the electrode unit 310 of the tip 300 through the handpiece 200.

Then, in step S82, the first control unit 120 and/or the second control unit 220 determine whether the operation for stop is detected (S82).

When the operation for stop is detected in the first control unit 120 and/or the second control unit 220 in step S82, the generation of high frequency energy is stopped at the energy generation unit 150 of the body 100 (S83).

In the process of determining whether the first mode or the second mode is detected (S13), if it is determined that the second mode is detected (S30), the first control unit 120 determines which of the first operation (i.e. a first operation selection) or the second operation (i.e., a second operation selection) is detected (S50).

If the first operation is detected in step S60, the first control unit 120 sets the function of the second control unit 220 of the handpiece 200 to the single irradiation operation (S61).

After the function of the second control unit 220 is set to the single irradiation operation (S61), whether the operation for start of the second control unit 220 is detected is determined (S80). In other words, in step S80, it is determined whether there is an input to start frequency energy generation.

When it is determined that the operation for start of the second control unit 220 is detected in step S80, high frequency energy is generated from the energy generation unit 150 of the body 100, and the multipolar-type output apparatus 10 irradiates the skin with the high frequency energy by outputting the high frequency energy to the electrode unit 310 of the tip 300 through the handpiece 200.

Then, in step S82, the first control unit 120 and/or the second control unit 220 determine whether the operation for stop is detected (S82).

When the operation for stop is detected in the first control unit 120 and/or the second control unit 220 in step S82, the generation of high frequency energy is stopped at the energy generation unit 150 of the body 100 (S83).

In the process of determining whether the first operation or the second operation is detected (S50), if the second operation (S70) is detected, a process of generating a small amount of high frequency energy from the energy generation unit 150 first and then irradiating the skin with the high frequency energy through the tip 300 so as to determine whether a return electrode is properly provided may be further included during the operation of generating high frequency energy from the energy generation unit 150 of the body 100 and outputting the high frequency energy to the electrode unit 310 of the tip 300 through the handpiece 200 so as irradiate the skin with the high frequency energy. At this time, the return electrode may electrically connected to the body 100, and a data value of the return electrode may be recognized by the first control unit 120. That is, a small amount of high frequency energy generated from the energy generation unit 150 of the body 100 is output through the tip 300, and the high frequency energy applied to the skin returns to the body 100 through the return electrode. At this time, whether the return electrode is properly provided may be determined on the basis of a resistance value input to the first control unit 120.

A process of displaying a warning window on the first display unit 130 may be further included when the resistance value input to the first control unit 120 exceeds 400Ω and so it is determined that the return electrode is not properly provided.

Although the embodiments of the present disclosure have been described above in detail, it will be understood that those skilled in the art to which the present disclosure pertains may implement the present disclosure in other various forms as well without departing from the technical spirit or essential features thereof. Also, it is noted that any one feature of an embodiment of the present disclosure described in the specification may be applied to another embodiment of the present disclosure. Similarly, the present invention encompasses any embodiment that combines features of one embodiment and features of another embodiment. Therefore, the scope of right of the present disclosure is not limited thereto, and various changes and modifications by those skilled in the art using the basic concept of the present disclosure defined by the following claims are also included in the scope of right of the present disclosure.

What is claimed is:

1. A multipolar-type output apparatus comprising:
a body configured to generate a high frequency energy;
a handpiece detachably attached to the body and configured to transmit the high frequency energy received from the body to the tip; and
a tip detachably attached to the handpiece and comprising an electrode unit configured to transmit the high frequency energy received from the handpiece to skin,
wherein the electrode unit comprises a first electrode and a second electrode, the first electrode and the second electrode having a size different from each other.

2. The multipolar-type output apparatus of claim 1,
wherein the tip comprises a contact surface at a portion of the tip that is brought into contact with the skin,
wherein the first electrode is in a protruding shape protruding from the contact surface of the tip, while the second electrode is in a flat shape.

3. The multipolar-type output apparatus of claim 2,
wherein the first electrode is in form of a needle to invade into the skin.

4. The multipolar-type output apparatus of claim 2,
wherein the first electrode is in form of a mild protrusion not to invade into the skin.

5. The multipolar-type output apparatus of claim 2,
wherein the first electrode comprises a plurality of first electrodes, each of which being in the protruding shape,
the second electrode, which is in the flat shape, is provided in an area surrounding the plurality of first electrodes,
while the plurality of first electrodes and the second electrode are distanced away from each other on the contact surface of the tip such that the plurality of first electrodes and the second electrode are not electrically connected on the contact surface of the tip.

6. The multipolar-type output apparatus of claim 2,
wherein the first electrode comprises a first group of multiple first electrodes and a second group of multiple first electrodes, and the second electrode comprises one or more second electrodes,
one of the one or more second electrodes is provided in an area surrounding the first group of multiple first electrodes, and the other of the one or more second electrodes is provided in an area surrounding the second group of multiple first electrodes.

7. The multipolar-type output apparatus of claim 1,
wherein the body comprises an energy generation unit configured to generate high frequency energy transmitted to the electrode unit and a body controller configured to control a polarity of the high frequency energy.

8. The multipolar-type output apparatus of claim 7,
wherein the body controller is operable to set the polarity of the high frequency energy in at least two different irradiation modes, in which the high frequency energy having different polarities depending on the at least two irradiation modes, according to a user input.

9. The multipolar-type output apparatus of claim 7,
wherein the first electrode comprises a first group of multiple first electrodes and a second group of multiple first electrodes, and the second electrode comprises one or more second electrodes,
wherein the body controller is operable to set the polarity of the high frequency energy in at least two different irradiation modes, wherein the at least two different irradiation modes comprises:
a first irradiation mode, in which the first group of multiple first electrodes have a positive (+) polarity while the second group of multiple first electrodes have a negative (−) polarity, and
a second irradiation mode, in which the first group of multiple first electrodes, the second group of multiple first electrodes, the one or more second electrodes have a same polarity, either positive (+) or negative (−).

10. The multipolar-type output apparatus of claim 9,
wherein, in the first irradiation mode, the one or more second electrodes are controlled not to irradiate with the high frequency energy.

11. The multipolar-type output apparatus of claim 1,
wherein the body comprises a cooling unit,
wherein the handpiece comprises
a gas transferring unit configured to receive a cooling gas generated from the cooling unit of the body and to transfer the cooling gas to the tip, and
wherein the tip comprises a chamber where the cooling gas supplied from the gas transferring unit stays when the cooling gas is output.

12. The multipolar-type output apparatus of claim 11,
wherein the body comprises a body controller and a storage storing a first identification value;
wherein the tip comprises
a second storage unit storing a second identification value of the tip,
wherein the body controller configures to determine matching of the first identification value and the second identification.

13. The multipolar-type output apparatus of claim 12,
wherein the handpiece comprises a handpiece controller configured to set operation of the handpiece in at least two different operation selections,
the at least two different operation selections comprise a first operation selection in which the electrode unit performs one time irradiation operation and a second operation selection in which the electrode unit performs continuous irradiation.

14. A control method for a multipolar-type output apparatus comprising a body, a handpiece, and a tip with an electrode unit, wherein the electrode unit comprises a plurality of first electrodes and one or more second electrodes, the one or more second electrodes having a different size from the plurality of first electrodes, the control method comprising:
determining, by a controller, whether a first irradiation mode or a second irradiation mode is detected,
a first irradiation mode, in which a first group of multiple first electrodes from among the plurality of first electrodes have a positive (+) polarity while a second group of multiple first electrodes from among the plurality of first electrodes have a negative (−) polarity, and
a second irradiation mode, in which the first group of multiple first electrodes, the second group of multiple first electrodes, the one or more second electrodes have a same polarity, either positive (+) or negative (−);
generating high frequency energy from an energy generation unit of the body and outputting the high frequency energy to the electrode unit of the tip through the handpiece so as to irradiate skin with the high frequency energy according to detected first irradiation mode or second irradiation mode.

15. The control method of claim 14, wherein the first irradiation mode is a high frequency energy transmission method in a bi-polar irradiation method, and the second irradiation mode is a high frequency energy transmission method in a mono-polar irradiation method.

16. The control method of claim 15, further comprising changing, by the controller, between a first operation selection and a second operation selection,
a first operation selection in which the electrode unit performs one time irradiation operation and a second operation selection in which the electrode unit performs continuous irradiation.

17. The control method of claim 16, further comprising generating a small amount of high frequency energy from the energy generation unit first and irradiating the skin with the small amount of high frequency energy so as to determine whether a return electrode is properly provided and outputting the high frequency energy to the electrode unit of the tip through the handpiece so as to irradiate the skin with the high frequency energy when it is determined that the return electrode is properly provided.

18. The control method of claim 17, wherein the return electrode is electrically connected to the body, and a data value of the return electrode is recognized by the controller.

19. The control method of claim 18, wherein whether the return electrode is properly provided is determined on the basis of a resistance value input to the controller.

20. The control method of claim 19, further comprising displaying a warning window on a display unit that the return electrode is not properly provided when the resistance value exceeds 400 Ω.
